(19) United States
(12) Patent Application Publication
(10) Pub. No.: US 2025/0213188 A1
(43) Pub. Date: Jul. 3, 2025

(54) BLOOD VESSEL VISUALIZATION DEVICE AND BLOOD VESSEL VISUALIZATION METHOD

(71) Applicant: TERUMO KABUSHIKI KAISHA, Tokyo (JP)

(72) Inventor: Shota TSUMAKI, Kai-shi (JP)

(73) Assignee: TERUMO KABUSHIKI KAISHA, Tokyo (JP)

(21) Appl. No.: 19/086,506

(22) Filed: Mar. 21, 2025

Related U.S. Application Data
(63) Continuation of application No. PCT/JP2023/034020, filed on Sep. 20, 2023.

(30) Foreign Application Priority Data
Sep. 22, 2022 (JP) 2022-151199

(51) Int. Cl. A61B 5/00

(52) U.S. Cl. CPC A61B 5/489; A61B 5/0077; A61B 5/0082; A61B 5/721; A61B 5/7267; A61B 2576/02

(57) ABSTRACT

A blood vessel visualization device for visualizing a blood vessel of a living body includes: an irradiation unit configured to irradiate a target site of the living body with light having a wavelength capable of passing through an inside of the living body; an imaging unit configured to capture an image of the target site irradiated with the light; a selection unit programmed to select, using the image of the target site captured by the imaging unit, a puncturing blood vessel site optimal for puncturing from among a plurality of the blood vessels; and a display unit configured to highlight at least one of the puncturing blood vessel site or a puncture position based on the puncturing blood vessel site on the target site.

### CROSS-REFERENCE TO RELATED APPLICATIONS

This is a bypass continuation of PCT Application No. PCT/JP2023/034020, filed on Sep. 20, 2023, which claims priority to Japanese Patent Application No. 20220151199, filed on Sep. 22, 2022. The entire contents of these applications are incorporated by reference herein.

### BACKGROUND

The present disclosure relates to a blood vessel visualization device and a blood vessel visualization method.

Japanese Patent No. 4555534 B2 and Japanese Patent No. 6127207 B2 disclose blood vessel visualization devices for visualizing blood vessels of a living body. The blood vessel visualization device includes an imaging unit and a display unit. The imaging unit images a target site of a living body. The display unit projects information on a blood vessel site inside the target site onto the target site using the image of the target site captured by the imaging unit.

### SUMMARY

However, in Japanese Patent No. 4555534 B2, when information on a blood vessel site is projected onto a target site, a shadow of a puncture needle puncturing the blood vessel site, a shadow caused by the shape of a living body, or the like may appear on the target site. A user such as a doctor or a nurse may erroneously recognize a shadow as information of the blood vessel site. In addition, when the puncture needle punctures the blood vessel site, information on the blood vessel site projected on the target site may be an obstacle.

An object of certain embodiments of the present disclosure is to solve the above-described problem.

(1) According to a first embodiment, a blood vessel visualization device for visualizing a blood vessel of a living body includes: an irradiation unit that irradiates a target site of the living body with light having a wavelength capable of passing through an inside of the living body; an imaging unit that captures an image of the target site irradiated with the light; a selection unit that selects a puncturing blood vessel site optimal for puncturing using an image of the target site captured by the imaging unit from among a plurality of the blood vessels; and a display unit that highlights at least one of the puncturing blood vessel site and a puncture position based on the puncturing blood vessel site on the target site.

According to this configuration, the puncturing blood vessel site or the puncture position is easily visually recognized. In addition, it is possible to prevent unnecessary information and light that hinder puncture from being displayed on the target site. As a result, the puncture position can be easily punctured, and the puncture success rate can be improved.

(2) In one aspect of the blood vessel visualization device according to (1), the blood vessel visualization device further includes: a puncturing blood vessel image generation unit that generates a puncturing blood vessel image obtained by visualizing the puncturing blood vessel site using the image, and the display unit highlights the puncturing blood vessel image on the target site.

According to this configuration, the puncturing blood vessel site can be easily visually recognized.

(3) In one aspect of the blood vessel visualization device according to (1) or (2), the display unit irradiates the puncture position based on the puncturing blood vessel site in the target site with a light beam.

According to this configuration, the puncture position can be easily visually recognized.

(4) In one aspect of the blood vessel visualization device according to any one of (1) to (3), the blood vessel visualization device further includes: an entire blood vessel image generation unit that generates an entire blood vessel image obtained by visualizing a plurality of the blood vessels existing inside the target site based on the image captured by the imaging unit, and the display unit displays the entire blood vessel image on the target site and then performs the highlighting.

According to this configuration, the user visually recognizes the puncturing blood vessel site or the puncture position after visually recognizing the entire blood vessel image. This eliminates the need to move the line of sight of the user. As a result, the puncturing blood vessel site or the puncture position can be more easily visually recognized, and the puncture position can be more easily punctured.

(5) In one aspect of the blood vessel visualization device according to (4), the display unit stops display of the entire blood vessel image when performing the highlighting.

According to this configuration, since only the puncturing blood vessel site or the puncture position is highlighted after the entire blood vessel image is displayed, the puncturing blood vessel site or the puncture position can be more easily visually recognized. As a result, the puncture position can be more easily punctured.

(6) In one aspect of the blood vessel visualization device according to any one of (1) to (5), the display unit ends the highlighting when a certain period of time elapses after the highlighting is performed.

According to this configuration, since the highlighting is automatically ended, the target site is easily visually recognized, and the puncture position is easily punctured.

(7) In one aspect of the blood vessel visualization device according to any one of (1) to (6), the blood vessel visualization device further includes: an image recognition unit that recognizes a position of a puncture needle based on the image, and the display unit ends the highlighting of the puncturing blood vessel site when the recognized puncture needle comes into contact with the puncturing blood vessel site or the puncture position.

According to this configuration, the puncture position can be reliably and efficiently punctured.

(8) In one aspect of the blood vessel visualization device according to any one of (1) to (7), the display unit highlights the puncture position on the target site when ending the highlighting of the puncturing blood vessel site.

According to this configuration, the user can easily specify the puncture position. As a result, the puncture position can be more easily punctured, and the puncture success rate can be further improved.

(9) In one aspect of the blood vessel visualization device according to any one of (1) to (8), the blood vessel visualization device includes: a motion detection unit that detects a motion vector of the living body based on the image, and the display unit uses the motion vector to perform the highlighting following a motion of the living body.

According to this configuration, even in a case where the living body moves, highlighting can be performed more accurately.

(10) In one aspect of the blood vessel visualization device according to any one of (1) to (9), the selection unit acquires at least one or more parameters of straightness, length, thickness, depth, and extension direction of a plurality of blood vessel sites of the target site based on the image, and selects a blood vessel site of which the acquired parameter is equal to or greater than a threshold as the puncturing blood vessel site.

According to this configuration, the puncturing blood vessel site can be accurately selected.

(11) In one aspect of the blood vessel visualization device according to (10), the selection unit: changes and re-selects the threshold in a case where there is a plurality of the selected puncturing blood vessel sites; and changes and re-selects the threshold in a case where there is no selected puncturing blood vessel site.

According to this configuration, since the puncturing blood vessel site can be appropriately selected, the puncture success rate is improved.

(12) In one aspect of the blood vessel visualization device according to any one of (1) to (9), the blood vessel visualization device includes: a machine learning model that uses the image as input data and outputs, as output data, puncture success rates of a plurality of blood vessel sites of the target site shown in the image, and the selection unit selects a blood vessel site having the puncture success rate higher than a threshold as the puncturing blood vessel site.

According to this configuration, the puncturing blood vessel site can be appropriately selected, and the puncture success rate can be further improved.

(13) According to a second embodiment, a blood vessel visualization method for visualizing a blood vessel of a living body includes: irradiating a target site of the living body with light having a wavelength capable of passing through an inside of the living body; capturing an image of the target site irradiated with the light; selecting a puncturing blood vessel site optimal for puncturing using an image of the target site captured by the imaging unit from among a plurality of the blood vessels; and highlighting at least one of the puncturing blood vessel site and a puncture position based on the puncturing blood vessel site on the target site.

According to the above configuration, the same effect as that of the first aspect can be obtained.

According to certain embodiments of the present disclosure, the puncturing blood vessel site or the puncture position is easily visually recognized. In addition, it is possible to prevent unnecessary information and light that hinder puncture from being displayed on the target site. As a result, the puncture position can be easily punctured, and the puncture success rate can be improved.

### BRIEF DESCRIPTION OF THE DRAWINGS

FIG. 1 is a configuration diagram of a blood vessel visualization device according to the present embodiment.

FIGS. 2A to 2C are diagrams illustrating arrangement of a camera and a first light source with respect to a target site.

FIG. 3A is a view illustrating imaging of a target site, and FIG. 3B is a view illustrating display of an entire blood vessel image on the target site.

FIG. 4A is a view illustrating highlighting of a puncturing blood vessel image on a target site, and FIG. 4B is a view illustrating highlighting of a puncturing blood vessel image and a puncture position on the target site.

FIG. 5A is a view illustrating highlighting of a puncture position to a target site, and FIG. 5B is a view illustrating puncture of a puncture needle to the puncture position.

FIG. 6 is a flowchart illustrating an operation of the blood vessel visualization device.

FIG. 7 is a flowchart illustrating an operation of the blood vessel visualization device.

FIG. 8 is a flowchart illustrating an operation of the blood vessel visualization device.

### DETAILED DESCRIPTION

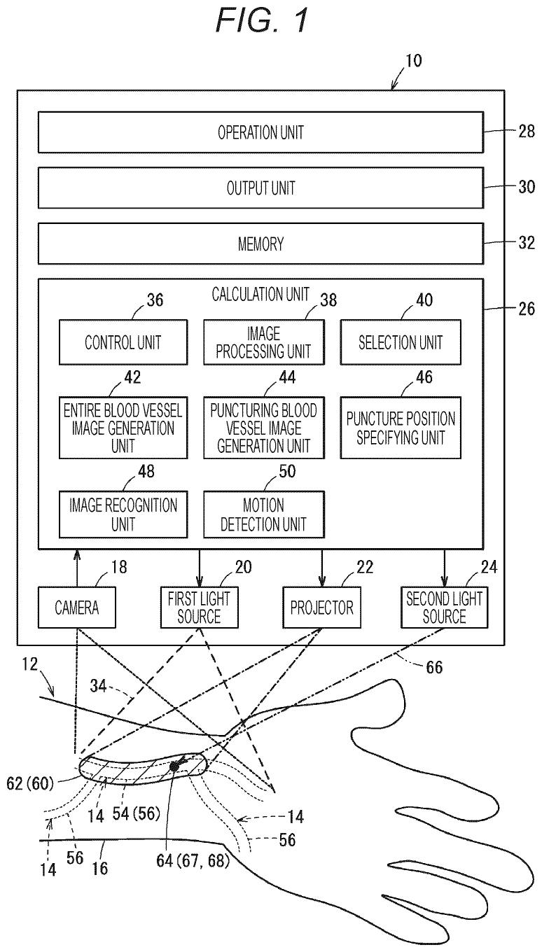

FIG. 1 is a configuration diagram of a blood vessel visualization device 10 according to the present embodiment. The blood vessel visualization device 10 visualizes a blood vessel 14 of a living body 12. Specifically, the blood vessel visualization device 10 visualizes a plurality of blood vessels 14 at a target site 16 of the living body 12. The living body 12 is, for example, a human body. The target site 16 is, for example, a hand, a wrist, a forearm, an upper arm, a foot, an ankle, a lower leg, or a thigh of a human body. In the following description, a case where the wrist of the human body is the target site 16 will be described.

The blood vessel visualization device 10 includes a camera 18 (imaging unit), a first light source 20 (irradiation unit), a projector 22 (display unit), a second light source 24 (display unit), a calculation unit 26, an operation unit 28, an output unit 30, and a memory 32.

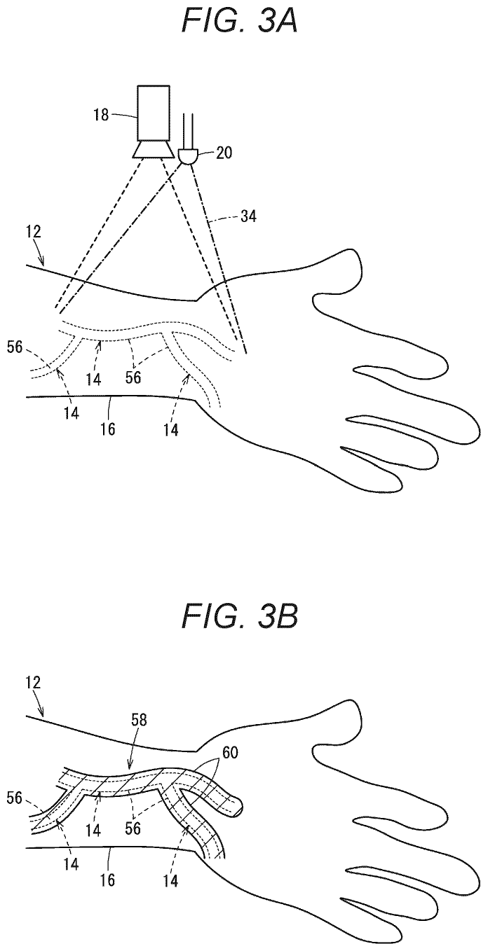

The first light source 20 irradiates the target site 16 of the living body 12 with light having a wavelength that can pass through the inside of the living body 12 (see FIGS. 1 and 3A). The first light source 20 is a light source that outputs near-infrared light 34. The first light source 20 outputs, for example, the near-infrared light 34 having a wavelength of 700 nm to 2500 nm. The first light source 20 can adjust the light amount of the near-infrared light 34. The first light source 20 includes one or a plurality of near-infrared light irradiation units.

The camera 18 captures an image of the target site 16 irradiated with the near-infrared light 34 (see FIGS. 1 and 3A). That is, the camera 18 can image the near-infrared light 34 emitted to the target site 16. The camera 18 outputs an image of the target site 16 including the near-infrared light 34 to the calculation unit 26. Note that the image of the target site 16 captured by the camera 18 is a moving image or a still image. In a case where a moving image is captured, the camera 18 continuously captures a moving image of the target site 16 including the near-infrared light 34. In a case where a still image is captured, the camera 18 captures a still image of the target site 16 including the near-infrared light 34 at a certain period of time intervals.

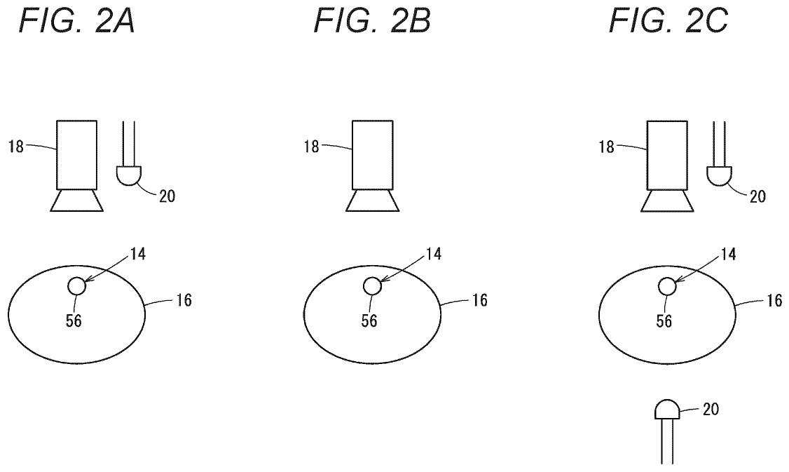

FIGS. 2A to 2C are diagrams illustrating arrangement of the camera 18 and the first light source 20 with respect to the target site 16. In FIG. 2A, the camera 18 and the first light source 20 are arranged to face the target site 16. In FIG. 2B, the camera 18 and the first light source 20 are arranged to face each other so as to sandwich the target site 16. In FIG. 2C, the camera 18 and the two first light sources 20 are arranged to face the target site 16. Specifically, in FIG. 2C, the camera 18 and one first light source 20 and the other first light source 20 are arranged to face each other so as to sandwich the target site 16. In any case of FIGS. 2A to 2C, the camera 18 can image the target site 16 including the near-infrared light 34 (see FIGS. 1 and 3A) with which the target site 16 is irradiated.

As illustrated in FIG. 1, the operation unit 28 is an operator that can be operated by a user. The operation unit 28 is, for example, an operation button provided in the blood vessel visualization device 10. The output unit 30 outputs various types of information regarding the target site 16. The output unit 30 is, for example, a display screen provided in the blood vessel visualization device 10.

The calculation unit 26 is a processor of the blood vessel visualization device 10. The calculation unit 26 executes a program stored in the memory 32 to implement the functions of a control unit 36, an image processing unit 38, a selection unit 40, an entire blood vessel image generation unit 42, a puncturing blood vessel image generation unit 44, a puncture position specifying unit 46, an image recognition unit 48, and a motion detection unit 50.

The control unit 36 controls each unit of the blood vessel visualization device 10.

The image processing unit 38 performs predetermined image processing on the image of the target site 16 captured by the camera 18. Examples of the image processing include adjustment of image parameters such as contrast and brightness. Specifically, the image processing unit 38 performs various types of image processing such as contrast enhancement, luminance leveling (histogram flattening), edge sharpening, sharpening, and gamma conversion on the image of the target site 16.

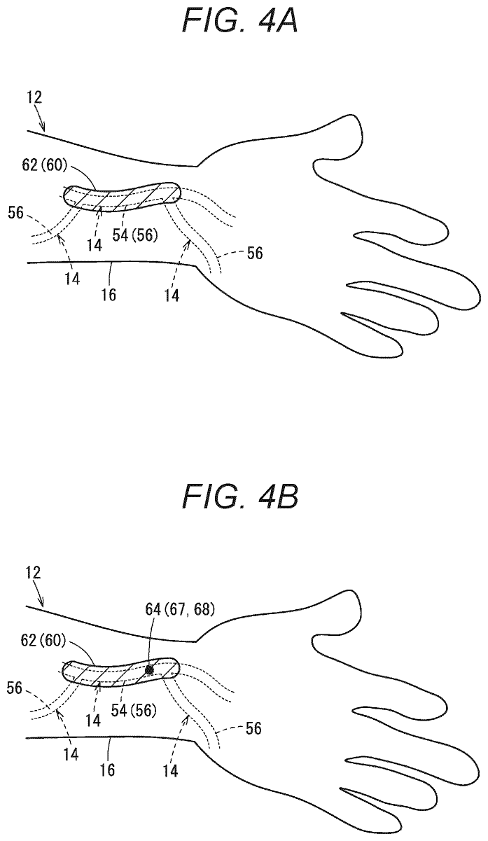

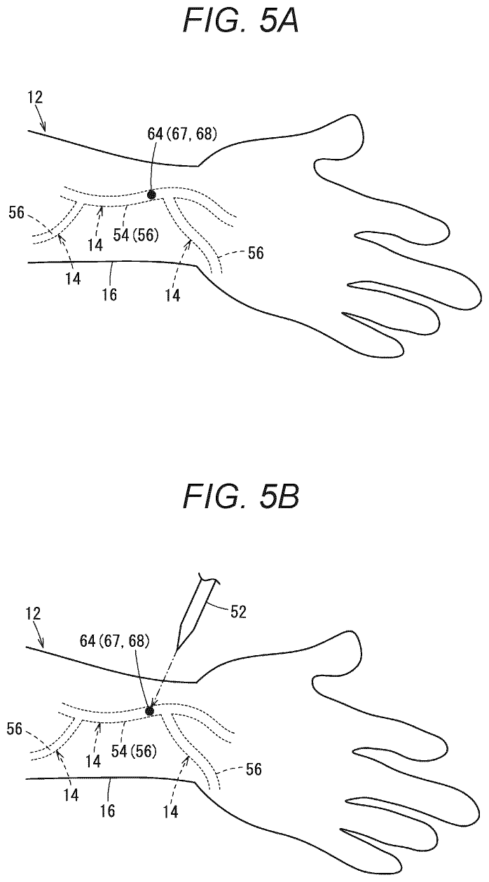

The selection unit 40 selects a puncturing blood vessel site 54 (see FIGS. 1 and 4A) optimal for puncturing the target site 16 by the puncture needle 52 (see FIG. 5B) using the image after the image processing from among the plurality of blood vessels 14 (see FIG. 3A) of the target site 16. The blood vessel site is a part of the blood vessel 14 existing in the living body 12. For example, in a case where one blood vessel 14 is branched from another blood vessel at a plurality of locations, the blood vessel site is a portion between two branch locations of the blood vessel 14. A blood vessel site 56 of the target site 16 is a part of the blood vessel 14 extending inside the target site 16 among the blood vessels 14 existing in the living body 12.

The selection unit 40 acquires at least one or more parameters of the straightness, the length, the thickness, the depth position (depth), and the matching degree between the extending direction and the extending direction of the target site 16 of the plurality of blood vessel sites 56 of the target site 16 on the basis of the image after the image processing. Among them, the straightness of the blood vessel site 56 is a degree of straightness of the blood vessel site 56. Furthermore, in the present specification, the depth position of the blood vessel site 56 is the position of the blood vessel site 56 with respect to the central portion inside the target site 16. In other words, the depth position of the blood vessel site 56 is a position indicating the shallowness of the blood vessel site 56 with respect to the skin of the target site 16.

The selection unit 40 selects the blood vessel site 56 in which the acquired parameter is equal to or greater than a threshold as the puncturing blood vessel site 54. The puncturing blood vessel site 54 is the blood vessel site 56 optimal for puncturing with a puncture needle 52. That is, the puncturing blood vessel site 54 is the blood vessel site 56 that is easily punctured by the puncture needle 52, and is also the blood vessel site 56 in which the puncture success rate of the puncture needle 52 is expected to be high. Note that the puncture success rate is a numerical value indicating the probability of success in puncturing the blood vessel site 56 with the puncture needle 52.

The straight blood vessel site 56 is easily punctured by the puncture needle 52. In addition, the relatively long blood vessel site 56 is easily punctured by the puncture needle 52. Furthermore, the relatively thick blood vessel site 56 is easily punctured by the puncture needle 52. In addition, among the plurality of blood vessel sites 56 of the target site 16, the blood vessel site 56 located near the skin of the target site 16 is easily punctured by the puncture needle 52. Furthermore, the blood vessel site 56 extending in the direction along the target site 16 is easily punctured by the puncture needle 52. If the puncture needle 52 punctures such a blood vessel site 56, the puncture success rate increases.

In addition, the selection unit 40 may add the past puncture history in the plurality of blood vessel sites 56 to the parameter. As a result, the selection unit 40 can select the blood vessel site 56 other than the blood vessel site 56 punctured in the past among the plurality of blood vessel sites 56 as the puncturing blood vessel site 54.

Furthermore, the selection unit 40 may add at least one of the sex of the living body 12, the presence or absence of the disease of the living body 12, the age of the living body 12, and the physical information such as BMI of the living body 12 to the parameter of the blood vessel site 56. The selection unit 40 can appropriately select the puncturing blood vessel site 54 in consideration of this parameter.

Furthermore, the selection unit 40 can change the parameter itself acquired from the image after the image processing. Specifically, as described above, the selection unit 40 can add the type of parameter acquired from the image after the image processing. In addition, the selection unit 40 can reduce the types of parameters acquired from the image after the image processing. As a result, the selection unit 40 can appropriately select the puncturing blood vessel site 54.

The target site 16 includes a plurality of blood vessel sites 56. Therefore, it is also conceivable that there is a plurality of blood vessel sites 56 equal to or larger than the threshold. In this case, the selection unit 40 may change (raise) the threshold and perform the selection again. That is, the selection unit 40 may make the selection stricter by changing the threshold. As a result, several blood vessel sites 56 are selected as candidates of the puncturing blood vessel site 54 in the first selection, and candidates of the plurality of puncturing blood vessel sites 54 are narrowed down in the second and subsequent selections, whereby a specific blood vessel site 56 can be appropriately selected (chosen) as the puncturing blood vessel site 54. As a result, the puncture success rate can be improved.

In this case, for example, when a large number of blood vessel sites 56 have been selected as candidates for the puncturing blood vessel site 54, the selection unit 40 may add BMI or the like to the parameter. As a result, since a large number of selected candidates of the puncturing blood vessel site 54 are narrowed down, the puncturing blood vessel site 54 can be appropriately selected.

In addition, it is also conceivable that none of the blood vessel sites 56 is equal to or greater than the threshold in the first selection, and the puncturing blood vessel site 54 cannot be selected. In this case, the selection unit 40 may change (lower) the threshold and perform the selection again. That is, the selection unit 40 may make the selection loose by changing the threshold. As a result, the specific blood vessel site 56 can be appropriately selected (chosen) as the puncturing blood vessel site 54 in the second and subsequent selections. As a result, the puncture success rate can be improved.

In this case, the selection unit 40 may extract the blood vessel site 56 equal to or greater than the threshold by deleting at least one parameter among the plurality of parameters. As a result, the puncturing blood vessel site 54 can be appropriately selected.

Furthermore, in a case where some blood vessel sites 56 are selected as candidates of the puncturing blood vessel site 54 in the first selection, the selection unit 40 may notify the user of the selected candidates of the plurality of puncturing blood vessel sites 54 by the output unit 30. Specifically, the selected candidates of the plurality of puncturing blood vessel sites 54 are displayed on the display screen of the output unit 30. The user selects a desired candidate from the plurality of candidates of the puncturing blood vessel site 54 by operating the operation button of the operation unit 28 while viewing the display content of the display screen. As a result, the selection unit 40 can select the candidate selected by the user as the puncturing blood vessel site 54.

Alternatively, the selection unit 40 may use artificial intelligence to select the puncturing blood vessel site 54. Specifically, a machine learning model is stored in the memory 32. This machine learning model is a machine learning model that uses the image after the image processing as input data and outputs the puncture success rate of the plurality of blood vessel sites 56 of the target site 16 shown in the image as output data. That is, the machine learning model is a model that learns the correlation between the parameter of the blood vessel site 56 based on the image after the image processing and the puncture success rate.

The selection unit 40 takes a machine learning model from the memory 32 and inputs the image after the image processing to the taken machine learning model. The machine learning model uses the input image to output the puncture success rate for each of the plurality of blood vessel sites 56 captured in the image. The selection unit 40 selects the blood vessel site 56 having a puncture success rate higher than the threshold as the puncturing blood vessel site 54.

Even in a case where the machine learning model is used, there may be a plurality of blood vessel sites 56 having a puncture success rate equal to or higher than the threshold, or the puncturing blood vessel site 54 may not be selected by one selection. In this case, the selection unit 40 may select the puncturing blood vessel site 54 by changing the threshold as described above. In addition, the selection unit 40 may select the puncturing blood vessel site 54 by changing (adding or deleting) the parameter itself as described above.

The entire blood vessel image generation unit 42 generates an entire blood vessel image 58 (see FIG. 3B) that visualizes the plurality of blood vessels 14 existing inside the target site 16 on the basis of the image after the image processing. In the entire blood vessel image 58, the central axis of each of a plurality of blood vessel images 60 may coincide with the central axis of the corresponding blood vessel 14. The thickness of each of the plurality of blood vessel images 60 may be substantially the same as the thickness of the corresponding blood vessel 14.

The puncturing blood vessel image generation unit 44 generates a puncturing blood vessel image 62 (see FIG. 4A) obtained by visualizing the puncturing blood vessel site 54 selected by the selection unit 40 using the image after the image processing. In this case, the puncturing blood vessel image generation unit 44 may extract the blood vessel image 60 corresponding to the puncturing blood vessel site 54 using the entire blood vessel image 58 generated by the entire blood vessel image generation unit 42, and may set the extracted blood vessel image 60 as the puncturing blood vessel image 62.

The puncture position specifying unit 46 specifies a puncture position 64 (see FIGS. 4B to 5B) of the puncture needle 52 in the target site 16 based on the puncturing blood vessel image 62.

The image recognition unit 48 recognizes the position of the puncture needle 52 on the basis of the image after the image processing. In a case where the puncture needle 52 is reflected in the image, the image recognition unit 48 determines whether the recognized puncture needle 52 comes into contact with the puncturing blood vessel site 54 or the puncture position 64.

The motion detection unit 50 detects a motion vector of the living body 12 on the basis of the moving image after the image processing.

The projector 22 displays the entire blood vessel image 58 on the target site 16 under the control of the control unit 36 (see FIG. 3B). Specifically, the projector 22 displays the entire blood vessel image 58 on the target site 16 by projecting the entire blood vessel image 58 on the target site 16.

In addition, the projector 22 highlights the puncturing blood vessel site 54 on the target site 16 under the control of the control unit 36 (see FIGS. 1 and 4A). Specifically, the projector 22 projects the puncturing blood vessel image 62 onto the target site 16 to highlight the puncturing blood vessel image 62 on the target site 16.

Under the control of the control unit 36, the second light source 24 irradiates the puncture position 64 of the target site 16 with a light beam 66 to highlight the puncture position 64 (see FIG. 4B). That is, the second light source 24 irradiates the puncture position 64 based on the puncturing blood vessel site 54 in the target site 16 with the light beam 66. As a result, a marking image 67 irradiated with the light beam 66 is formed at the puncture position 64.

The second light source 24 is, for example, a laser irradiator that outputs the light beam 66 that is visible light of 400 nm to 700 nm. The second light source 24 is a laser irradiator such as an LD or an LED. Therefore, the second light source 24 emits the light beam 66 having directivity and a small orientation angle. In this case, the size of the irradiation range (marking image 67) of the light beam 66 in the target site 16 is desirably equal to or smaller than the thickness of the puncturing blood vessel site 54 and the thickness of the puncturing blood vessel image 62. As a result, it is possible to avoid that the puncture of the puncture position 64 by the puncture needle 52 is prevented.

The second light source 24 can adjust the luminance of the light beam 66 (marking image 67) according to the surrounding environment. Further, the marking image 67 may be various patterns such as a straight line such as a dotted line or a solid line, a polygon such as a circle or a triangle, and a sign such as X.

The control unit 36 controls the projector 22 and the second light source 24 to display the entire blood vessel image 58, highlight the puncturing blood vessel site 54, and highlight the puncture position 64 (marking image 67) as follows.

The projector 22 displays the entire blood vessel image 58 on the target site 16 (see FIG. 3B), and then highlights the puncturing blood vessel image 62 (see FIG. 4A). In addition, the second light source 24 highlights the puncture position 64 when the projector 22 highlights the puncturing blood vessel image 62 (see FIG. 4B). In this case, the projector 22 stops displaying the entire blood vessel image 58 and highlights the puncturing blood vessel image 62. That is, when the puncturing blood vessel image 62 is highlighted, the display of the entire blood vessel image 58 is stopped.

After the puncturing blood vessel image 62 and the puncture position 64 are highlighted, the control unit 36 stops the operation of the projector 22. As a result, the highlighting of the puncturing blood vessel image 62 is stopped, and only the highlighting of the puncture position 64 is performed (see FIG. 5A).

In this case, when a certain period of time has elapsed since the start of the highlighting of the puncturing blood vessel image 62 or the highlighting of the puncture position 64, the control unit 36 ends the highlighting of the puncturing blood vessel image 62. Alternatively, when the image recognition unit 48 determines that the puncture needle 52 comes into contact with the puncturing blood vessel site 54 or the puncture position 64, the control unit 36 ends the highlighting of the puncturing blood vessel image 62. As a result, the target site 16 can be easily visually recognized, and thus the puncture by the puncture needle 52 can be easily performed (see FIG. 5B).

In addition, the control unit 36 may end each highlighting when a certain period of time has elapsed after at least one of the highlighting of the puncturing blood vessel image 62 and the highlighting of the puncture position 64 is performed. Alternatively, when the image recognition unit 48 determines that the puncture needle 52 comes into contact with the puncturing blood vessel site 54 or the puncture position 64, the control unit 36 may end each highlighting. Even in this case, since the target site 16 is easily visually recognized, the puncture by the puncture needle 52 is easily performed.

Furthermore, the camera 18 may capture the target site 16 while the puncturing blood vessel image 62 is highlighted and the puncture position 64 is highlighted. As a result, it is possible to grasp whether the marking image 67 is shifted from the puncturing blood vessel image 62 on the basis of the image captured by the camera 18.

Furthermore, when the motion detection unit 50 detects the motion of the living body 12, the control unit 36 may cause the projector 22 and the second light source 24 to perform highlighting following the motion of the living body 12. For example, by moving the projector 22 and the second light source 24 following the motion of the living body 12 using a moving mechanism (not illustrated), it is possible to continuously perform highlighting on the target site 16. Alternatively, by changing the projection direction of the projector 22 and the irradiation direction of the light beam 66 following the motion of the living body 12, it is possible to continuously perform highlighting on the target site 16.

In the above description, the second light source 24 highlights the puncture position 64. In the blood vessel visualization device 10, the projector 22 may highlight the puncturing blood vessel site 54 and highlight the puncture position 64. In this case, the puncture position specifying unit 46 generates a puncture position image 68 indicating the puncture position 64. The projector 22 highlights at least one of the puncturing blood vessel image 62 and the puncture position image 68 on the target site 16 under the control of the control unit 36.

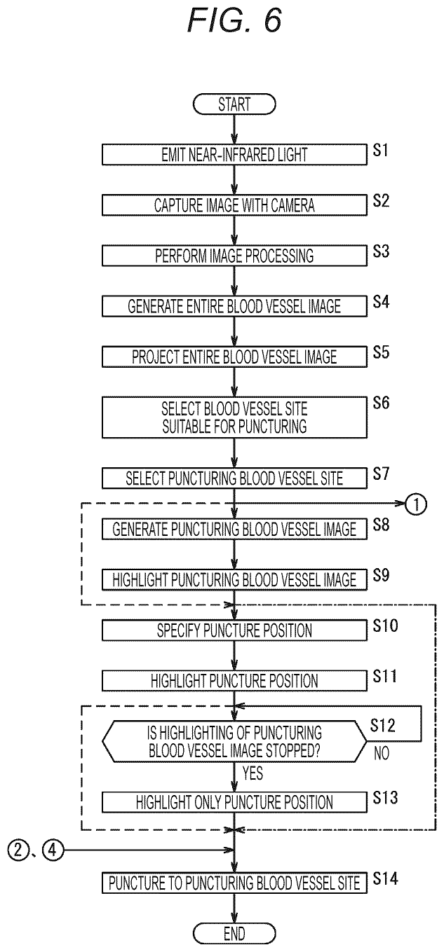

Next, an operation (blood vessel visualization method) of the blood vessel visualization device 10 will be described with reference to flowcharts of FIGS. 6 to 8.

In step S1, the first light source 20 irradiates the target site 16 with the near-infrared light 34 (see FIGS. 1 and 3A).

In step S2, the camera 18 captures an image of the target site 16 irradiated with the near-infrared light 34. The camera 18 outputs the image of the target site 16 to the calculation unit 26.

In step S3, the image processing unit 38 performs image processing on the input image of the target site 16.

In step S4, the entire blood vessel image generation unit 42 generates the entire blood vessel image 58 (see FIG. 3B) based on the image after the image processing.

In step S5, the projector 22 projects the entire blood vessel image 58 on the target site 16 under the control of the control unit 36.

In step S6, the selection unit 40 selects a blood vessel site 56 suitable for puncturing the target site 16 by the puncture needle 52 (see FIG. 5B) using the image after the image processing from among the plurality of blood vessel sites 56 of the target site 16. In this case, the selection unit 40 selects a blood vessel site 56 in which the parameter related to the blood vessel site 56 is equal to or more than the threshold. Alternatively, the selection unit 40 uses the machine learning model to select the blood vessel site 56 in which the puncture success rate is equal to or greater than the threshold.

In step S7, the selection unit 40 selects, as the puncturing blood vessel site 54, a blood vessel site 56 optimal for puncturing the target site 16 by the puncture needle 52 among the plurality of selected blood vessel sites 56. In this case, the selection unit 40 changes the threshold and selects again the blood vessel site 56 whose parameter is equal to or greater than the threshold, thereby selecting one blood vessel site 56 as the puncturing blood vessel site 54. Alternatively, the selection unit 40 selects the blood vessel site 56 having the highest puncture success rate as the puncturing blood vessel site 54.

In a case where only one blood vessel site 56 is selected in step S6, the selection unit 40 may select one blood vessel site 56 as the puncturing blood vessel site 54 in step S7.

In step S8, the puncturing blood vessel image generation unit 44 generates the blood vessel image 60 corresponding to the puncturing blood vessel site 54 as the puncturing blood vessel image 62 (see FIG. 4A) using the entire blood vessel image 58.

In step S9, the projector 22 projects the puncturing blood vessel image 62 onto the target site 16 under the control of the control unit 36 to highlight the puncturing blood vessel image 62.

In step S10, the puncture position specifying unit 46 specifies the puncture position 64 (see FIGS. 4B to 5B) of the puncture needle 52 in the target site 16 on the basis of the puncturing blood vessel image 62.

In step S11, the second light source 24 performs highlighting of the puncture position 64 by irradiating the puncture position 64 of the target site 16 with the light beam 66 to form the marking image 67 under the control of the control unit 36.

In step S12, the control unit 36 determines whether to stop the highlighting of the puncturing blood vessel image 62.

When a certain period of time has elapsed from the start of the highlighting in step S9 or step S11, the control unit 36 determines to stop the highlighting of the puncturing blood vessel image 62 (step S12: YES). Alternatively, when the image recognition unit 48 determines that the puncture needle 52 comes into contact with the puncturing blood vessel site 54 or the puncture position 64, the control unit 36 determines to stop the highlighting of the puncturing blood vessel image 62 (step S12: YES). Thereafter, the blood vessel visualization device 10 proceeds to step S13.

In step S13, the projector 22 stops the highlighting of the puncturing blood vessel image 62 under the control of the control unit 36. As a result, only the puncture position 64 is highlighted by the second light source 24.

In step S14, the user punctures the puncturing blood vessel site 54 with the puncture needle 52 by puncturing the highlighted puncture position 64 (marking image 67) by the puncture needle 52. As a result, the puncture needle 52 can easily puncture the puncturing blood vessel site 54.

In the blood vessel visualization device 10, the process in FIG. 6 can be changed as follows.

In the blood vessel visualization device 10, as indicated by a broken line in FIG. 6, it is also possible to skip the processes of steps S8, S9, S12, and S13. In this case, the puncturing blood vessel image 62 is not highlighted, and only the puncture position 64 is highlighted by the second light source 24. Even in this case, the puncture position 64 can be easily punctured by the puncture needle 52.

In addition, the blood vessel visualization device 10 can skip the processes of steps S10 to S13 as indicated by a one-dot chain line in FIG. 6. In this case, the puncture position 64 is not highlighted, and only the puncturing blood vessel image 62 is highlighted by the projector 22. Even in this case, the puncture position 64 can be easily punctured by the puncture needle 52.

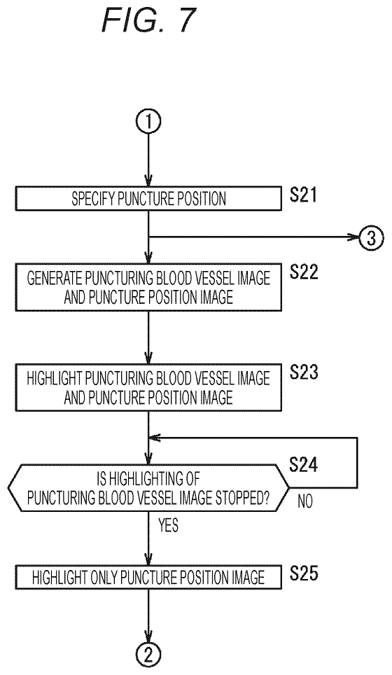

Furthermore, the blood vessel visualization device 10 may perform the processes of steps S21 to S25 illustrated in FIG. 7 instead of steps S8 to S13. Steps S21 to S25 are the processes in a case where the projector 22 highlights the puncturing blood vessel site 54 and highlights the puncture position 64.

After step S7, in step S21, the puncture position specifying unit 46 specifies the puncture position 64 of the target site 16 as in step S10 (see FIG. 6).

In step S22, the puncturing blood vessel image generation unit 44 generates the puncturing blood vessel image 62 as in step S8. In addition, the puncture position specifying unit 46 generates a puncture position image 68 indicating the specified puncture position 64.

In step S23, the projector 22 projects the puncturing blood vessel image 62 and the puncture position image 68 on the target site 16 under the control of the control unit 36 to highlight the puncturing blood vessel image 62 and the puncture position image 68 (see FIG. 4B).

In step S24, the control unit 36 determines whether to stop the highlighting of the puncturing blood vessel image 62 as in step S12.

In a case where the control unit 36 determines to stop the highlighting of the puncturing blood vessel image 62 (step S24: YES), the blood vessel visualization device 10 proceeds to step S25.

In step S25, the projector 22 stops the highlighting of the puncturing blood vessel image 62 under the control of the control unit 36. As a result, only the puncture position image 68 is highlighted (see FIG. 5A). Thereafter, the process in step S14 of FIG. 6 is performed.

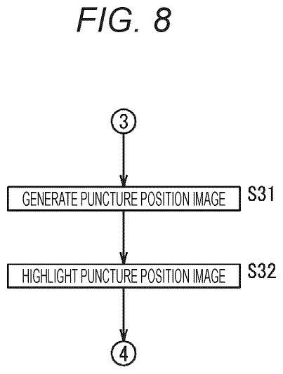

In addition, the blood vessel visualization device 10 may perform the processes of steps S31 and S32 illustrated in FIG. 8 instead of steps S22 to S25. Steps S31 and S32 are the processes in a case where the projector 22 performs only the highlighting of the puncture position 64.

After step S21, in step S31, the puncture position specifying unit 46 generates the puncture position image 68 as in step S22 (see FIG. 7).

In step S32, the projector 22 projects only the puncture position image 68 onto the target site 16 under the control of the control unit 36 to highlight only the puncture position image 68 (see FIG. 5A). Thereafter, the process in step S14 of FIG. 6 is performed.

Also in each process of FIGS. 7 and 8, the puncture position 64 and the puncturing blood vessel site 54 can be easily punctured with the puncture needle 52.

Note that the present invention is not limited to the above disclosure, and various configurations can be adopted without departing from the gist of the present invention.

1. A blood vessel visualization device for visualizing a blood vessel of a living body, the blood vessel visualization device comprising:
an irradiation unit configured to irradiate a target site of the living body with light having a wavelength capable of passing through an inside of the living body;
an imaging unit configured to capture an image of the target site irradiated with the light;
a selection unit programmed to select, using the image of the target site captured by the imaging unit, a puncturing blood vessel site optimal for puncturing from among a plurality of the blood vessels; and
a display unit configured to highlight at least one of the puncturing blood vessel site or a puncture position based on the puncturing blood vessel site on the target site.

2. The blood vessel visualization device according to claim 1, further comprising:
a puncturing blood vessel image generation unit that generates a puncturing blood vessel image obtained by visualizing the puncturing blood vessel site using the image, wherein:
the display unit highlights the puncturing blood vessel image on the target site.

3. The blood vessel visualization device according to claim 1, wherein the display unit is configured to irradiate the puncture position based on the puncturing blood vessel site in the target site with a light beam.

4. The blood vessel visualization device according to claim 1, further comprising:
an entire blood vessel image generation unit programmed to generate an entire blood vessel image obtained by visualizing a plurality of the blood vessels existing inside the target site based on the image captured by the imaging unit, wherein:
the display unit is configured to display the entire blood vessel image on the target site and subsequently perform the highlighting.

5. The blood vessel visualization device according to claim 4, wherein the display unit is configured to stop display of the entire blood vessel image when performing the highlighting.

6. The blood vessel visualization device according to claim 1, wherein the display unit is configured to end the highlighting when a predetermined period of time elapses after the highlighting is performed.

7. The blood vessel visualization device according to claim 1, further comprising:
an image recognition unit programmed to recognize a position of a puncture needle based on the image, wherein:
the display unit is configured to end the highlighting of the puncturing blood vessel site when the recognized puncture needle comes into contact with the puncturing blood vessel site or the puncture position.

8. The blood vessel visualization device according to claim 1, wherein the display unit is configured to initially highlight at least the puncturing blood vessel site, and subsequently end the highlighting of the puncturing blood vessel site and begin or continue highlighting the puncture position.

9. The blood vessel visualization device according to claim 1, comprising:
a motion detection unit configured to detect a motion vector of the living body based on the image, wherein:
the display unit is configured to use the motion vector to perform the highlighting following a motion of the living body.

10. The blood vessel visualization device according to claim 1, wherein the selection unit is programmed to acquire at least one or more parameters of straightness, length, thickness, depth, and extension direction of a plurality of blood vessel sites of the target site based on the image, and to select a blood vessel site of which the acquired parameter is equal to or greater than a threshold as the puncturing blood vessel site.

11. The blood vessel visualization device according to claim 10, wherein:
the selection unit is programmed to:
change and re-select the threshold in a case where there are multiple selected puncturing blood vessel sites of which the acquired parameter is equal to or greater than a threshold, and
change and re-select the threshold in a case where there is no selected puncturing blood vessel site of which the acquired parameter is equal to or greater than the threshold.

12. The blood vessel visualization device according to claims 1, comprising:
a machine learning model programmed to use the image as input data and to output, as output data, puncture success rates of a plurality of blood vessel sites of the target site shown in the image, wherein:
the selection unit is programmed to select a blood vessel site having a puncture success rate higher than a threshold as the puncturing blood vessel site.

13. A blood vessel visualization method for visualizing a blood vessel of a living body, the blood vessel visualization method comprising:
irradiating a target site of the living body with light having a wavelength capable of passing through an inside of the living body;
capturing an image of the target site irradiated with the light;
selecting, using an image of the target site captured by the imaging unit, a puncturing blood vessel site optimal for puncturing from among a plurality of the blood vessels; and
highlighting at least one of the puncturing blood vessel site or a puncture position based on the puncturing blood vessel site on the target site.

14. A blood vessel visualization device for visualizing a blood vessel of a living body, the blood vessel visualization device comprising:
a first light source configured to irradiate a target site of the living body with light having a wavelength capable of passing through an inside of the living body;
an camera configured to capture an image of the target site irradiated with the light;
a processor programmed to select, using the image of the target site captured by the camera, a puncturing blood vessel site optimal for puncturing from among a plurality of the blood vessels; and
at least one of a projector configured to highlight the puncturing blood vessel site on the target site, or a second light source configured to highlight a puncture position based on the puncturing blood vessel site on the target site.
